(19) United States
(12) Patent Application Publication
(10) Pub. No.: US 2012/0219625 A1
(43) Pub. Date: Aug. 30, 2012

(54) CLARITHROMYCIN EXTENDED-RELEASE TABLET

(76) Inventor: Bernard Charles Sherman, Toronto (CA)

(21) Appl. No.: 13/394,035

(22) PCT Filed: Sep. 2, 2010
(86) PCT No.: PCT/CA10/01373

§ 371 Date: May 14, 2012

Related U.S. Application Data
(60) Provisional application No. 61/239,210, filed on Sep. 2, 2009.

(51) Int. Cl. A61K 9/28; A61P 31/04; A61K 31/7048

(52) U.S. Cl. 424/468; 514/29

(57) ABSTRACT

The present application relates to an extended-release tablet for oral administration of Clarithromycin. The tablet comprises a water soluble diluent and an acidic compound wherein the acidic compound is used to increase the dissolution rate of Clarithromycin. The tablet is free from polymers or may comprise polymer that is insufficient to function as a release controlling agent.

### TECHNICAL FIELD

The present invention relates to an extended-release tablet for oral administration of the antibiotic Clarithromycin.

### BACKGROUND

Known in the art is that polymers are used as the mechanisms to control the release of Clarithromycin. For example, U.S. Pat. No. 6,010,718 discloses an extended release tablet comprising Clarithromycin and a pharmaceutically acceptable polymer. The patent specifically teaches seven polymers that serve to slow the dissolution of the tablet in gastrointestinal fluid, to effect extended-release, and enable reduction of variation in Clarithromycin blood levels and reduction of adverse effects, while maintaining bioavailability (i.e. extent of absorption) substantially equivalent to that of the immediate-release Clarithromycin tablets. Those specific polymers are: polyvinylpyrrolidine, hydroxypropyl cellulose, hydroxypropylmethyl cellulose, methyl cellulose, vinyl acetate/crotonic acid copolymers, methacrylic acid copolymers, maleic anhydride-methyl vinyl ether copolymers and derivatives and mixtures thereof. The patent also teaches that the polymer must be water-soluble and hydrophilic.

Polymers are either water soluble or water insoluble. Water soluble polymers generally form viscous solutions at low concentrations. Hence, water soluble polymers when used to control release of a drug generally do so by forming a viscous gel at the surface of a tablet. Because of its high viscosity, the viscous gel dissolves away slowly. On the other hand, water insoluble polymers (or insoluble waxes) are used to delay release by forming insoluble matrices from which the active ingredient will diffuse only slowly.

### SUMMARY

In illustrative embodiments of the present invention, there is provided an extended-release Clarithromycin tablet without a polymer.

Clarithromycin has relatively low solubility in water. Consequently, a tablet that comprises Clarithromycin and a water soluble filler such as sorbitol, without any disintegrant will dissolve too slowly to enable full release of the Clarithromycin after ingestion. Some embodiments of the present invention provide a tablet that comprises, Clarithromycin, a water soluble diluent, and an acidic compound. The acidic compound is sufficiently acidic and present in sufficient quantity to increase the dissolution rate, but not so acidic as to significantly detract from the stability of the composition.

Illustrative embodiments of the present invention provide an extended-release tablet for oral administration comprising Clarithromycin, a water soluble diluent and an acidic compound, wherein the acidic compound is operable to increase a rate of dissolution of Clarithromycin.

Illustrative embodiments of the present invention provide a tablet described herein wherein the tablet is free from polymers or the only polymer is in a film coating that surrounds the tablet.

Illustrative embodiments of the present invention provide a tablet described herein wherein the tablet further comprises a polymer in an amount that is insufficient to function as a Clarithromycin release controlling polymer.

Illustrative embodiments of the present invention provide a tablet described herein wherein the tablet comprises a film coating that surrounds the tablet.

Illustrative embodiments of the present invention provide a tablet described herein wherein the acidic compound is selected from the group consisting of: potassium bitartrate, sodium bitartrate and monosodium citrate.

Illustrative embodiments of the present invention provide a tablet described herein wherein the acidic compound is potassium bitartrate.

Illustrative embodiments of the present invention provide a tablet described herein wherein the acidic compound is present in an amount of from about 5% to about 20% of the tablet by weight.

Illustrative embodiments of the present invention provide a tablet described herein wherein the water soluble diluent comprises sorbitol.

Illustrative embodiments of the present invention provide a tablet described herein wherein the water soluble diluent consists of sorbitol.

Illustrative embodiments of the present invention provide a tablet described herein wherein the water soluble diluent is present in an amount of from about 15% to about 45% of the tablet by weight.

Illustrative embodiments of the present invention provide a tablet described herein wherein the tablet exhibits dissolution from about 50% to about 80% at 6 hours.

Other aspects and features of the present invention will become apparent to those ordinarily skilled in the art upon review of the following description of specific embodiments of the invention.

### DETAILED DESCRIPTION

In illustrative embodiments, the present invention provides an extended-release Clarithromycin tablet without a polymer.

In illustrative embodiments, the present invention provides a tablet that comprises, Clarithromycin, a water soluble diluent, and an acidic compound. The acidic compound is sufficiently acidic and present in sufficient quantity to increase the dissolution rate, but not so acidic as to significantly detract from the stability of the composition.

Suitable acidic compounds may include, for example and without limitation, potassium bitartrate, sodium bitartrate and monosodium citrate. Often, potassium bitartrate is present in tablets of the present invention.

Acidic compounds suitable for use in the tablets of the present invention do not form a viscous solution as the tablet dissolves and hence do not form a gel at the surface of the tablet. Acidic compounds suitable for use in this invention are not insoluble and thus do not form an insoluble matrix. Acidic compounds suitable for use in the present invention serve to increase the dissolution rate of the Clarithromycin, as the solubility of Clarithromycin increases with increased acidity.

An amount of acidic compound used in tablets of the present invention may be from about 5% to about 20% by weight. Often the amount of acidic compound in tablets of the present invention is about 8%.

Water soluble diluents suitable for use in tablets of the present invention include, without limitation, lactose and sorbitol. Often, sorbitol is present in tablets of the present invention. Water soluble diluents may be present in tablets of the present invention in an amount of from about 15% to about 45% by weight.

Tablets of the present invention are often tablets that comprise a lubricant. An example of a suitable lubricant is, without limitation, magnesium stearate.

While not required, some embodiments of tablets of the present invention may comprise a polymer used for a purpose other than controlling release of Clarithromycin. In such embodiments, the polymer will not significantly change a rate of release of Clarithromycin. For example, tablets of the present invention may comprise a polymer that would be capable of significantly changing the rate of release of the Clarithromycin if present at above a threshold concentration, but such polymers may only be present in tablets of the present invention at concentrations below the threshold concentration. For another example, tablets of the present invention may comprise a polymer that is incapable of significantly changing the rate of release of Clarithromycin and hence such a polymer may be present in the tablet in any quantity. Such threshold concentrations will vary from polymer to polymer. The controlled release characteristics of polymers in this regard is well understood in the art and persons of skill in the art readily know what the threshold concentration is for a particular polymer.

Tablets of the present invention may be film-coated. The film coat may be used to cover the foul taste of Clarithromycin and/or for improved elegance. Such film coatings may dissolve rapidly and will not significantly affect the release of the active ingredient.

Tablets can be measured for their dissolution rate by using a standardized testing protocol well understood to a person of skill in the art. Tablets of the present invention, when tested for dissolution in USP apparatus 2 at 75 rpm in 900 mL of 0.05 M phosphate buffer pH6.8, often exhibit a dissolution of from about 50% to about 80% at 6 hours.

### EXAMPLES

The following examples are illustrative of some of the embodiments of the invention described herein. These examples should not be considered to limit the spirit or scope of the invention in any way.

### Example 1

Tablets were made with ingredients per tablet as follows:

| | |
|---|---|
| Clarithromycin | 500.0 mg |
| Sorbitol | 257.5 mg |
| Potassium Bitartrate | 67.5 mg |
| Magnesium Stearate | 35.0 mg |
| Total | 860.0 mg |

The process of manufacture was to mix the ingredients, compact, mill the compacted material into granules, and recompress the granules into tablets having a weight of 860 mg per tablet.

When tested for dissolution in USP apparatus 2 at 75 rpm in 900 mL of 0.5 M phosphate buffer pH 6.8, the mean results were about 70% at 6 hours.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes one such thing in addition to more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. An extended-release tablet for oral administration comprising Clarithromycin, a water soluble diluent and an acidic compound, wherein the acidic compound is operable to increase a rate of dissolution of Clarithromycin.

2. The tablet of claim 1 wherein the tablet is free from polymers or the only polymer is in a film coating that surrounds the tablet.

3. The tablet of claim 1 wherein the tablet further comprises a polymer in an amount that is insufficient to function as a Clarithromycin release controlling polymer.

4. The tablet of claim 1 wherein the tablet comprises a film coating that surrounds the tablet.

5. The tablet of claim 1 wherein the acidic compound is selected from the group consisting of: potassium bitartrate, sodium bitartrate and monosodium citrate.

6. The tablet of claim 1 wherein the acidic compound is potassium bitartrate.

7. The tablet of claim 1 wherein the acidic compound in an amount of from about 5% to about 20% of the tablet by weight.

8. The tablet of claim 1 wherein the water soluble diluent comprises sorbitol.

9. The tablet of claim 1 wherein the water soluble diluent consists of sorbitol.

10. The tablet of claim 1 wherein the water soluble diluent is present in an amount of from about 15% to about 45% of the tablet by weight.

11. The tablet of claim 1 wherein the tablet exhibits dissolution from about 50% to about 80% at 6 hours.

12. The tablet of claim 3 wherein the tablet comprises a film coating that surrounds the tablet.

13. The tablet of claim 12 wherein the acidic compound is selected from the group consisting of: potassium bitartrate, sodium bitartrate and monosodium citrate.

14. The tablet of any one of claims 13 wherein the acidic compound is potassium bitartrate.

15. The tablet of claim 14 wherein the acidic compound in an amount of from about 5% to about 20% of the tablet by weight.

16. The tablet of claim 15 wherein the water soluble diluent comprises sorbitol.

17. The tablet of claim 16 wherein the water soluble diluent consists of sorbitol.

18. The tablet of claim 17 wherein the water soluble diluent is present in an amount of from about 15% to about 45% of the tablet by weight.

19. The tablet of claim 18 wherein the tablet exhibits dissolution from about 50% to about 80% at 6 hours.
